(19) United States
(12) Patent Application Publication
(10) Pub. No.: US 2026/0134994 A1
(43) Pub. Date: May 14, 2026

(54) INFORMATION PROCESSING SYSTEM, INFORMATION PROCESSING METHOD, AND PROGRAM

(71) Applicant: I'mbesideyou Inc., Tokyo (JP)

(72) Inventors: Shozo KAMIYA, Tokyo (JP); Kotaro ANDO, Tokyo (JP)

(21) Appl. No.: 19/212,682

(22) Filed: May 20, 2025

(30) Foreign Application Priority Data
Nov. 13, 2024 (JP) 2024-198648

(51) Int. Cl. G16H 50/20; G06T 7/00; G16H 10/20

(52) U.S. Cl. CPC G16H 50/20; G06T 7/0012; G16H 10/20; G06T 2207/10016; G06T 2207/20081; G06T 2207/30004

(57) ABSTRACT

An information processing system for assisting in the differentiation of mental disorders. The system comprises: an answer input unit configured to accept an answer from a patient to a question for differentiating a mental disorder; a video image acquisition unit configured to acquire a video image capturing the patient; a biometric reaction detection unit configured to analyze the video image to detect a change in biometric reaction of the patient; and an estimation unit configured to estimate the mental disorder that the patient suffers from by inputting the accepted answer and the detected change in biometric reaction into a learning model, wherein the learning model is trained on the answer, the change in biometric reaction, and the mental disorder.

### TECHNICAL FIELD

The present invention relates to an information processing system, an information processing method, and a program.

### BACKGROUND OF THE INVENTION

Patent Literature 1 evaluates cardiovascular disease risk.

### PRIOR ART

Patent Literature 1: Japanese Unexamined Patent Application Publication No. 2022-136784

### SUMMARY OF THE INVENTION

It is difficult to determine a patient's subjective condition such as mental health.

The present invention has been made in view of such background and aims to provide a technology capable of supporting the differentiation of mental disorders.

The principal invention for solving the above problem is an information processing system comprising: an answer input unit configured to accept an answer from a patient to a question for differentiating a mental disorder; a video image acquisition unit configured to acquire a video image capturing the patient; a biometric reaction detection unit configured to analyze the video image to detect a change in biometric reaction of the patient; and an estimation unit configured to estimate the mental disorder that the patient suffers from by inputting the accepted answer and the detected change in biometric reaction into a learning model, wherein the learning model is trained on the answer, the change in biometric reaction, and the mental disorder.

Other problems disclosed in this application and their solutions will be made clear in the embodiment section and drawings.

According to the present invention, it is possible to support the differentiation of mental disorders.

### BRIEF DESCRIPTION OF DRAWINGS

FIG. 1 shows an example of the overall configuration of the information processing system.

FIG. 2 shows an example of the hardware configuration of the management server 2.

FIG. 3 shows an example of the software configuration of the management server 2.

FIG. 4 is a diagram explaining the operation of the management server 2.

### DETAILED DESCRIPTION OF THE INVENTION

### System Overview

The following describes an information processing system according to an embodiment of the present invention. The information processing system of this embodiment supports the differential diagnosis of mental disorders (depression, bipolar disorder, obsessive-compulsive disorder, sleep disorder, eating disorder, alcohol dependence, adjustment disorder, autism spectrum disorder, attention deficit hyperactivity disorder, schizophrenia, dementia, developmental disorder, panic disorder, PTSD, etc.). The information processing system of this embodiment differentiates mental disorders (or normal condition) based on a patient's answers to questions for differentiating mental disorders (tests such as QIDS, DSM-IV, DSM-5, ADOS-2, ASRS, EAT-26, MMSE, etc.) and the analysis results of a video image capturing the patient's conversation behavior (daily conversation).

Conversations in medical settings can be broadly divided into specialized conversations for examination and diagnosis (conversations conducted as part of medical practice, such as confirming symptoms, explaining test results, discussing treatment plans) and daily conversational exchanges (casual conversations in waiting rooms, small talk about weather or family updates before and after examinations, natural dialogues not for diagnostic purposes). In this embodiment, daily conversation refers to linguistic communication as a social interaction that naturally occurs between people present, not conducted for specific duties or purposes. Daily conversation is characterized by low purposefulness (not primarily aimed at specific problem-solving or information gathering), spontaneity (not planned), bidirectionality (mutual interaction rather than one-way information transmission), and functions to maintain/build social relationships. In other words, among linguistic communications occurring in the same medical setting, conversations for medical interviews or diagnoses are excluded from “daily conversation,” while casual conversations exchanged between examinations are included in “daily conversation.” Note that daily conversation includes situations where the patient is speaking one-sidedly (medical professionals or agents are mainly listening to the patient).

Additionally, the information processing system of this embodiment accepts input of differentiation results from a physician (including non-physician medical professionals; the same applies hereinafter) and determines the validity of the physician's differentiation results by comparing them with the differentiation results based on the answers and video image.

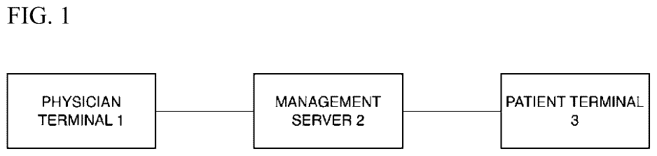

FIG. 1 shows an example of the overall configuration of the information processing system. The information processing system of this embodiment includes a management server 2. The management server 2 is connected to a physician terminal 1 and a patient terminal 3 via a communication network to enable communication. The communication network is, for example, the Internet, and is constructed using public telephone networks, mobile phone networks, wireless communication channels, Ethernet (registered trademark), etc.

The physician terminal 1 is a computer operated by a physician. The physician terminal 1 can be, for example, a smartphone, a tablet computer, a personal computer, etc.

The patient terminal 3 is a computer operated by a patient. The patient terminal 3 can be, for example, a smartphone, a tablet computer, a personal computer, etc.

The management server 2 may be a general-purpose computer such as a workstation or a personal computer, or it may be logically implemented through cloud computing.

### Management Server

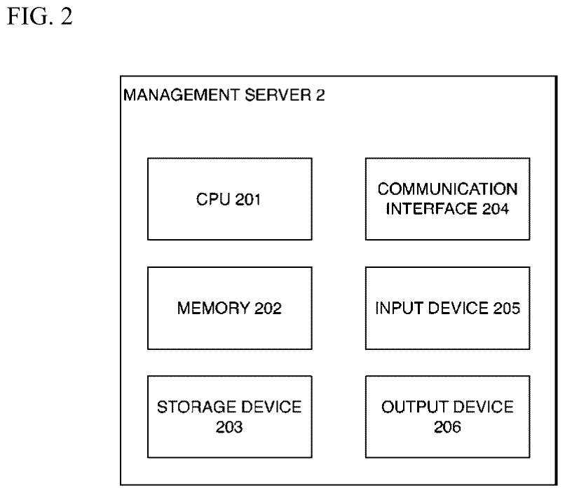

FIG. 2 shows an example of the hardware configuration of the management server 2. Note that the illustrated configuration is an example and may have configurations other than this. The management server 2 includes a CPU 201, memory 202, storage device 203, communication interface 204, input device 205, and output device 206. The storage device 203 stores various data and programs, and is, for example, a hard disk drive, solid-state drive, flash memory, etc. The communication interface 204 is an interface for connecting to a communication network, and is, for example, an adapter for connecting to Ethernet (registered trademark), a modem for connecting to a public telephone network, a wireless communication device for wireless communication, a USB (Universal Serial Bus) connector or RS232C connector for serial communication, etc. The input device 205 inputs data, and is, for example, a keyboard, mouse, touch panel, button, microphone, etc. The output device 206 outputs data, and is, for example, a display, printer, speaker, etc. Each functional unit of the management server 2 described later is realized by the CPU 201 reading and executing a program stored in the storage device 203 into the memory 202, and each storage unit of the management server 2 is realized as part of the storage area provided by the memory 202 and the storage device 203.

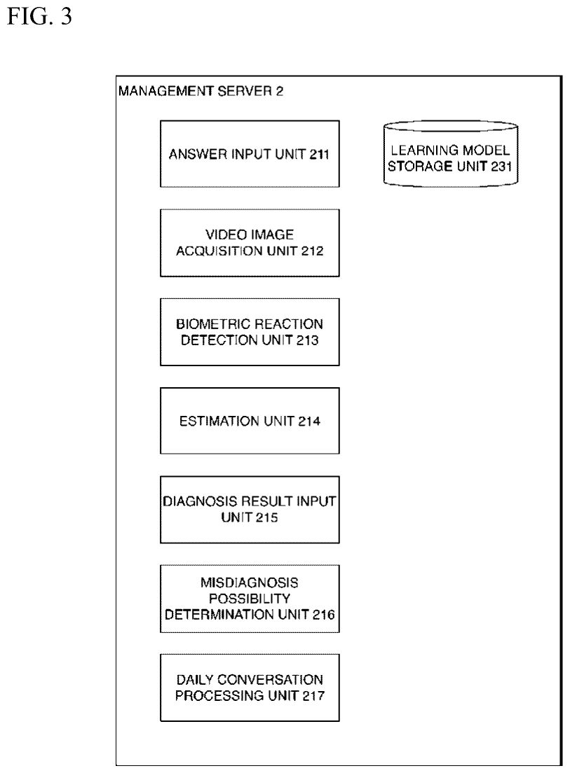

FIG. 3 shows an example of the software configuration of the management server 2. The management server 2 includes an answer input unit 211, a video image acquisition unit 212, a biometric reaction detection unit 213, an estimation unit 214, a diagnosis result input unit 215, a misdiagnosis possibility determination unit 216, a daily conversation processing unit 217, and a learning model storage unit 231.

### Storage Units

The learning model storage unit 231 stores a learning model trained by machine learning for estimating mental disorders (hereinafter referred to as a “disorder model”). The disorder model stored in the learning model storage unit 231 may be, for example, one trained by machine learning using training data consisting of a patient's answers to questions for differentiating mental disorders, changes in the patient's biometric reactions, and the mental disorder the patient suffers from (or the fact that the patient is healthy). The disorder model can be a classifier. The disorder model may also be a generator (large language model) with fine-tuning. The learning model storage unit 231 may be provided by an external server rather than by the management server 2, allowing the management server 2 to access the external server.

The learning model storage unit 231 may also store a learning model trained by machine learning for determining a physician's diagnosis result (hereinafter referred to as a “determination model”). The determination model can be created by machine learning using training data consisting of the estimation result of a mental disorder using the disorder model, the diagnosis result by a physician, and the correctness of that diagnosis result. The determination model may also be created using training data consisting of a patient's answers to questions for differentiating mental disorders, changes in the patient's biometric reactions, and the diagnosis result by a physician. Also, attributes of the physician (which may be information identifying the physician, or the physician's age group, years of experience, main specialty, etc.) may be given as features to the determination model. In this case, the management server 2 may include a physician information storage unit that manages physician attributes, and during inference, read the attributes of the diagnosing physician from the physician information storage unit and provide them to the determination model.

### Functional Units

The answer input unit 211 accepts input of a patient's answer to a question for differentiating a mental disorder. The answer input unit 211 may accept input of a patient's answer from a physician (receive the answer from the physician terminal 1), or it may present questions to the patient and directly accept input of answers from the patient (receive answers from the patient terminal 3).

The video image acquisition unit 212 acquires a video image capturing the patient. The video image is assumed to capture the patient engaged in daily conversation. The video image acquisition unit 212 can acquire a video image capturing the conversation between the patient and the daily conversation processing unit 217 described later. The video image acquisition unit 212 can receive a video image of the patient's behavior captured by the patient terminal 3 using a camera. The video image acquisition unit 212 may also acquire a video image capturing the patient's conversation behavior from the physician terminal 1. In this case, the physician terminal 1 may perform the recording, or a video file recorded by a separate camera may be sent from the physician terminal 1 to the management server 2.

The biometric reaction detection unit 213 analyzes the video image to detect changes in the patient's biometric reactions.

For example, the biometric reaction detection unit 213 can separate the video image into a set of images (collection of frame images) and audio, and analyze changes in biometric reactions from each. For example, the biometric reaction detection unit 213 can analyze the user's facial image using frame images separated from the video image to analyze changes in biometric reactions related to at least one of facial expressions, gaze, pulse, and facial movements. Also, the biometric reaction detection unit 213 can analyze the audio separated from the video image to analyze changes in biometric reactions related to at least one of the user's speech content and voice quality.

When a person's emotions change, they appear as changes in biometric reactions such as facial expressions, gaze, pulse, facial movements, speech content, and voice quality. In this embodiment, by analyzing changes in the user's biometric reactions, changes in the user's emotions are analyzed. An example of emotions analyzed in this embodiment is the degree of pleasure/displeasure. In this embodiment, the biometric reaction detection unit 213 can calculate a biometric reaction index value that reflects the content of changes in biometric reactions by numerically quantifying the changes in biometric reactions according to a predetermined standard.

The analysis of changes in facial expressions is performed, for example, as follows. For each frame image, the face region is identified from the frame image, and the facial expression identified in that region is classified into multiple categories according to a pre-trained image analysis model. Then, based on the classification results, an analysis is made as to whether a positive facial expression change is occurring between consecutive frame images, whether a negative facial expression change is occurring, and how large a facial expression change is occurring, and a facial expression change index value can be calculated according to the analysis results.

The analysis of changes in gaze is performed, for example, as follows. For each frame image, the eye region is identified from the frame image, and the direction of both eyes is analyzed to determine where the user is looking. For example, it analyzes whether the user is looking at the displayed speaker's face, the displayed shared materials, or outside the screen. It may also analyze whether the movement of the gaze is large or small, whether the frequency of movement is high or low, etc. Changes in gaze are also related to the user's level of concentration. The biometric reaction detection unit 213 can calculate a gaze change index value according to the results of the gaze change analysis.

The analysis of changes in pulse is performed, for example, as follows. For each frame image, the face region is identified from the frame image. Then, using a pre-trained image analysis model that captures numerical values of facial color information (G of RGB), changes in G color on the face surface are analyzed. By arranging the results along the time axis, a waveform representing changes in color information is formed, and the pulse is identified from this waveform. People's pulse rates increase when they are tense and decrease when they calm down. The biometric reaction analysis unit 213 can calculate a pulse change index value according to the results of the pulse change analysis.

The analysis of changes in facial movements is performed, for example, as follows. For each frame image, the face region is identified from the frame image, and the direction of the face is analyzed to determine where the user is looking. For example, it analyzes whether the user is looking at the displayed speaker's face, the displayed shared materials, or outside the screen. It may also analyze whether the movement of the face is large or small, whether the frequency of movement is high or low, etc. The facial movement and gaze movement may be analyzed together. For example, it may analyze whether the user is looking straight at the displayed speaker's face, looking up or down at it, or looking at it from an angle. The biometric reaction analysis unit 213 can calculate a face direction change index value according to the results of the facial direction change analysis.

The analysis of speech content is performed, for example, as follows. The biometric reaction analysis unit 213 converts the audio of a specified time (e.g., about 30-150 seconds) into text by performing well-known speech recognition processing, and performs morphological analysis on the text to remove unnecessary words for expressing conversation, such as particles and articles. Then, the remaining words are vectorized, and an analysis is made as to whether a positive emotional change is occurring, whether a negative emotional change is occurring, and how large an emotional change is occurring, and a speech content index value can be calculated according to the analysis results.

The analysis of voice quality is performed, for example, as follows. The biometric reaction unit 12, by performing well-known voice analysis processing on the audio of a specified time (e.g., about 30-150 seconds), identifies the acoustic features of the voice. Then, based on these acoustic features, an analysis is made as to whether a positive voice quality change is occurring, whether a negative voice quality change is occurring, and how large a voice quality change is occurring, and a voice quality change index value can be calculated according to the analysis results.

The biometric reaction analysis unit 213 calculates a biometric reaction index value using at least one of the facial expression change index value, gaze change index value, pulse change index value, face direction change index value, speech content index value, and voice quality change index value calculated as described above. For example, the biometric reaction index value can be calculated by weighted calculation of the facial expression change index value, gaze change index value, pulse change index value, face direction change index value, speech content index value, and voice quality change index value.

The estimation unit 214 estimates the patient's mental disorder. The estimation unit 214 can estimate the mental disorder based on the patient's answer and the change in biometric reaction (biometric reaction index value). The estimation unit 214 can estimate the mental disorder that the patient suffers from by inputting the accepted answer and the detected change in biometric reaction into the disorder model stored in the learning model storage unit 231.

Furthermore, the estimation unit 214 can determine the probability that the patient suffers from each of multiple mental disorders based on the confidence of the inference by the disorder model. Specifically, it uses the membership probabilities for each mental disorder class calculated in the output layer of the disorder model. Normally, the disorder model receives input data (patient's answers and biometric reaction index values) and probabilistically outputs which mental disorder class that data belongs to. For example, probabilities of suffering from multiple mental disorders are calculated, such as a 70% probability of depression, a 20% probability of bipolar disorder, and a 10% probability of schizophrenia. The confidence level may also be used as a “probability.” That is, the non-linear possibility of suffering may be called a probability.

The estimation unit 214 can evaluate the accuracy of the diagnosis based on the estimated probabilities of mental disorders. The reliability of the estimation result can be evaluated by the absolute value of the probability of suffering from the mental disorder with the highest probability. For example, if the probability of depression is 90% or higher, it can be determined that there is a very high possibility of depression, while if the probability of the mental disorder with the highest probability is around 50%, it can be determined that the accuracy of the diagnosis is not so high.

Also, the estimation unit 214 can suggest the possibility of comorbidities based on the estimated probabilities of mental disorders. If the probabilities of suffering from two or more mental disorders are both high, it may suggest the possibility that these disorders coexist.

The diagnosis result input unit 215 accepts the result of a differential diagnosis of a patient's mental disorder by a physician. The differential diagnosis by the physician is based on the answers above. The diagnosis result input unit 215 can receive the diagnosis result from the physician terminal 1.

The misdiagnosis possibility determination unit 216 determines the possibility of misdiagnosis in the physician's differentiation result accepted by the diagnosis result input unit 215. The misdiagnosis possibility determination unit 216 can determine the possibility of misdiagnosis by whether the mental disorder estimation result by the estimation unit 214 matches the diagnosis result accepted by the diagnosis result input unit 215. The misdiagnosis possibility determination unit 216 can determine the probability of misdiagnosis according to the probabilities of mental disorders estimated by the estimation unit 214.

The misdiagnosis possibility determination unit 216 may infer the possibility of misdiagnosis (or the possibility of correct diagnosis) by providing the mental disorder estimation result by the estimation unit 214 and the diagnosis result by the physician to the determination model stored in the learning model storage unit 231. The misdiagnosis possibility determination unit 216 may infer the possibility of misdiagnosis (or the possibility of correct diagnosis) by providing the patient's answers, the detected changes in biometric reactions, and the physician's diagnosis result to the determination model stored in the learning model storage unit 231.

The misdiagnosis possibility determination unit 216 can send the possibility of misdiagnosis to the physician terminal 1 to warn the physician of the possibility of misdiagnosis. The misdiagnosis possibility determination unit 216 may also send the possibility of a physician's misdiagnosis to a terminal of a manager of the medical institution, etc. In this case, an administrator storage unit that stores information indicating an administrator, etc., for each physician can be provided, and when the possibility of misdiagnosis is above a certain value, or regardless of the degree of possibility of misdiagnosis, the administrator, etc., corresponding to the physician can be read from the administrator storage unit, and the possibility of misdiagnosis can be sent to the read administrator. The misdiagnosis possibility determination unit 216 may also send the possibility of misdiagnosis to the patient terminal 3 to notify the patient.

The daily conversation processing unit 217 conducts conversations (daily conversations) with the patient. The daily conversation processing unit 217 can, for example, realize natural conversation with the patient by generating appropriate responses according to the patient's speech content using a large language model. The large language model used in this embodiment is a model that can understand the context and meaning of language by learning a large amount of text data and can generate natural text like a human according to the given context. Specifically, large language models such as the GPT (Generative Pre-trained Transformer) series, BERT (Bidirectional Encoder Representations from Transformers) series, XLNet, ELMO (Embeddings from Language Models), etc., can be used.

The daily conversation processing unit 217 has the large language model generate speech content to be conveyed to the patient according to the patient's speech content (which can be obtained by analyzing the audio extracted from the video image received from the patient terminal 3). Specifically, the following procedure is considered:
(1) Convert the patient's speech content to text data.
(2) Tokenize the converted text data to fit the input format for the large language model.
(3) Input the tokenized data into the large language model and generate an appropriate response text according to the context.
(4) Convert the generated response text into audio data using a speech synthesis engine and output it to the patient.

The daily conversation processing unit 217 can, for example, generate conversation content to be conveyed to the patient by giving a prompt to the large language model that includes the history of the patient's speech content (may be all or a specified number of the most recent ones) and an instruction to generate speech content to be conveyed to the patient according to that speech content. An example of a prompt could be as follows:
Prompt example:
   The following is a conversation history with a patient.
Patient: “Hello.”
System: “Hello. It's warm today, isn't it?”
Patient: “Ah, yes. It might be warm.”
Please consider what the system should say to the patient next.

The daily conversation processing unit 217 conducts such conversations with the patient and can obtain useful information about the patient's mental state by analyzing the content. The daily conversation processing unit 217 can also obtain more detailed information by analyzing not only the content of the patient's responses but also the response time, word choice in responses, speech speed, and intonation.

### Operation

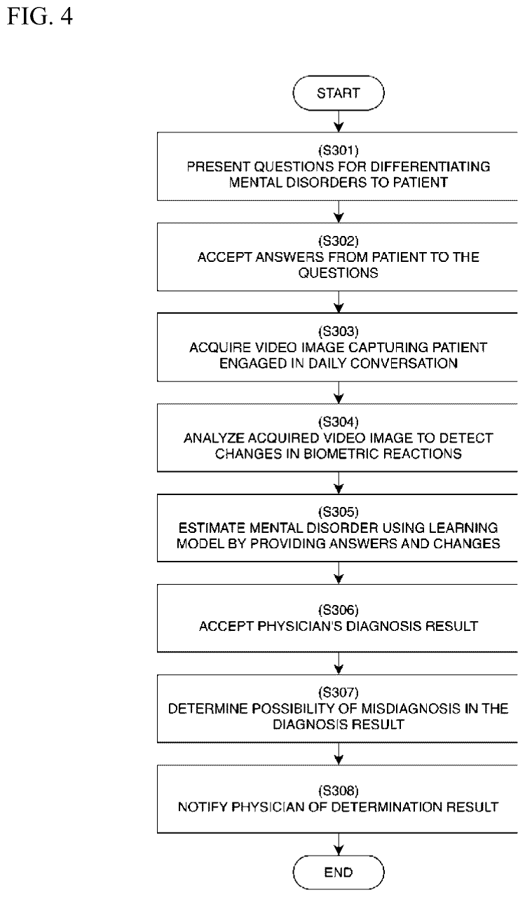

FIG. 4 is a diagram explaining the operation of the management server 2.

The management server 2 presents questions for differentiating mental disorders to the patient (S301), accepts answers from the patient to the questions (S302), acquires a video image capturing the patient engaged in daily conversation (S303), analyzes the acquired video image to detect changes in biometric reactions (S304), and estimates the mental disorder using the learning model by providing the accepted answers and detected changes in biometric reactions (S305). Additionally, the management server 2 can accept a physician's diagnosis result (S306), determine the possibility of misdiagnosis in that diagnosis result (S307), and notify the physician of the determination result (S308).

As described above, the information processing system of this embodiment can differentiate a patient's mental disorder from a video image capturing the patient's behavior in addition to answers to questions. It is known that when experts observe the behavior of people suffering from mental disorders, they can infer mental disorders even without specifically asking test questions, and by adding judgment from video images, improvement in the accuracy of inference is expected. Also, even for patients who intentionally give answers different from their actual condition to questions, it is expected that the possibility of mental disorders can be correctly estimated by also performing differentiation from video images.

Also, in the information processing system of this embodiment, the possibility of misdiagnosis can be determined based on the physician's diagnosis result and the computer's inference result.

The above embodiment has been described to facilitate understanding of the present invention and is not intended to limit the interpretation of the present invention. The present invention can be changed and improved without departing from its spirit, and the present invention includes its equivalents.

For example, the processing by each functional unit of the management server 2 described above may be executed by any functional unit. Also, a different functional unit that executes part of the processing of the functional units described above may be added. Also, the functional units of the management server 2 may be distributed across multiple computers.

Also, the information stored in each storage unit of the management server 2 may be stored in any storage unit. That is, the information stored in multiple storage units described above may be stored by a single storage unit, or part of the information stored in one storage unit described above may be stored by another storage unit.

### Variation Example 1

By setting biometric reaction indicators that should be particularly emphasized for each type of mental disorder and focusing on analyzing changes in those indicators, more accurate diagnostic support can be provided. Below are examples of biometric reaction indicators that should be emphasized in the diagnosis of representative mental disorders.

### (1) Depression

In the diagnosis of depression, it is effective to emphasize the following biometric reaction indicators:
Facial expression: Facial muscle movements are poor, and expressions of sadness or pain are often observed.
Gaze: The gaze is directed downward, and the frequency of eye contact decreases.
Voice quality: Voice inflection is poor, tending to be monotonous and quiet.

### (2) Bipolar Disorder

In the diagnosis of bipolar disorder, it is effective to emphasize the following biometric reaction indicators:
Facial expression: (Hypomanic state) Facial muscle movements are active, and expressions of joy or excitement are often observed. (Depressive state) Facial muscle movements are poor, and expressions of sadness or pain are often observed.
Gaze: (Hypomanic state) The gaze is directed upward, and the frequency of eye contact increases. (Depressive state) The gaze is directed downward, and the frequency of eye contact decreases.
Voice quality: (Hypomanic state) Voice inflection is rich, tending to be loud. (Depressive state) Voice inflection is poor, tending to be monotonous and quiet.

### (3) Schizophrenia

In the diagnosis of schizophrenia, it is effective to emphasize the following biometric reaction indicators:
Facial expression: Due to flattening of affect, changes in facial expressions become poor.
Gaze: The gaze may fixate on one point or, conversely, may not settle.
Speech content: Disjointed content reflecting decreased reality testing is common.

### (4) Panic Disorder

In the diagnosis of panic disorder, it is effective to emphasize the following biometric reaction indicators:
Pulse: Marked tachycardia is observed during panic attacks.
Facial expression: Expressions of strong fear are observed during panic attacks.
Voice quality: During panic attacks, the voice tends to tremble and words get stuck.

### (5) Dementia

In the diagnosis of dementia, it is effective to emphasize the following biometric reaction indicators:
Speech content: Statements about time and place become inaccurate, reflecting memory disorders and disorientation.
Voice quality: Speech rate tends to slow down, and voice volume tends to decrease.
Facial expression: Due to flattening of affect, changes in facial expressions become poor.

Note that the biometric reaction indicators exemplified above are merely representative and the biometric reaction indicators themselves are just examples.

### Variation Example 2

Patient attributes such as age and gender can be considered when estimating mental disorders or determining the possibility of misdiagnosis. Examples of attribute information that should be considered during estimation and determination are shown below.

### (1) Age

The prevalence and manifestation of symptoms of mental disorders may differ by age. For example, dementia is common in the elderly and rare in young people. Also, autism spectrum disorder in childhood may show behavioral characteristics different from those in adults. Therefore, by considering the patient's age, it is possible to evaluate using diagnostic criteria more appropriate for that age group.

The estimation unit 214 may provide the patient's age as one of the input information to the disorder model stored in the learning model storage unit 231. This enables estimation of mental disorders according to age.

The misdiagnosis possibility determination unit 216 may provide the patient's age as one of the input information to the determination model stored in the learning model storage unit 231. This enables determination of the possibility of misdiagnosis considering age.

### (2) Gender

It is known that some mental disorders show gender differences in prevalence and symptoms. For example, the lifetime prevalence of depression is higher in women, and the lifetime prevalence of alcohol dependence is higher in men. Also, eating disorders are more common in women. Therefore, by considering the patient's gender, it is possible to make diagnoses that take into account symptoms and prevalence characteristic to gender.

The estimation unit 214 may provide the patient's gender as one of the input information to the disorder model stored in the learning model storage unit 231. This enables estimation of mental disorders according to gender.

The misdiagnosis possibility determination unit 216 may provide the patient's gender as one of the input information to the determination model stored in the learning model storage unit 231. This enables determination of the possibility of misdiagnosis considering gender.

### (3) Medical History

The patient's history of mental disorders or physical illnesses has a significant impact on the interpretation and diagnosis of current symptoms. For example, if a patient with a history of depression again presents with depressive symptoms, it is more likely to be a recurrence of depression rather than a simple stress response. Also, a history of thyroid disease or cerebrovascular disease can cause psychiatric symptoms. Therefore, by considering the patient's medical history, more appropriate diagnoses can be made.

The estimation unit 214 may provide the patient's medical history as one of the input information to the disorder model stored in the learning model storage unit 231. This enables estimation of mental disorders based on medical history.

The misdiagnosis possibility determination unit 216 may provide the patient's medical history as one of the input information to the determination model stored in the learning model storage unit 231. This enables determination of the possibility of misdiagnosis considering medical history.

The information processing system of this embodiment may include an attribute information storage unit that manages the patient's attributes such as age, gender, and medical history. The estimation unit 214 and the misdiagnosis possibility determination unit 216 can read necessary attribute information from the attribute information storage unit and use it for estimation and determination processing.

Also, attribute information may be input by physicians or patients, or it may be automatically extracted from medical questionnaires or medical records.

### Variation Example 3

When the misdiagnosis possibility determined by the misdiagnosis possibility determination unit 216 is high, various methods can be used to alert physicians and patients.

### (1) Display of Alert Messages on Physician Terminal

The misdiagnosis possibility determination unit 216 may display an alert message on the physician terminal 1 when it determines that the possibility of misdiagnosis is high. The alert message can include the fact that there is a high possibility of misdiagnosis and the reason for it (e.g., the disorder estimated from the patient's answers and biometric reactions differs from the physician's diagnosis). The alert message may also include wording that encourages reconsideration or suggests additional tests or interviews.

### (2) Inclusion of Misdiagnosis Possibility in Diagnosis Report

The misdiagnosis possibility determination unit 216 may automatically include the fact that there is a high possibility of misdiagnosis in the diagnosis report created by the physician when it determines that the possibility of misdiagnosis is high. This allows the physician to recognize the possibility of misdiagnosis when reviewing the diagnosis report. The misdiagnosis possibility information included in the diagnosis report can also be used for determining subsequent treatment policies and sharing information with other medical staff.

### (3) Sending Alert Messages to Administrators

The misdiagnosis possibility determination unit 216 may send an alert message to the administrator of the medical institution or the supervisor of the department when it determines that the possibility of misdiagnosis is high. This enables management of diagnosis quality and, if necessary, guidance and support for the physician. The alert message can include the fact that there is a high possibility of misdiagnosis, the reason for it, and information such as the name of the relevant physician and patient.

### (4) Display of Alert Messages on Patient Terminal

The misdiagnosis possibility determination unit 216 may display an alert message on the patient terminal 3 when it determines that the possibility of misdiagnosis is high. The alert message can convey that the current diagnosis result needs reconsideration and include content encouraging additional tests or interviews with the physician. However, careful consideration must be given to the content and expression of the message to avoid causing anxiety to the patient.

### (5) Recording and Analysis of Alert History

The misdiagnosis possibility determination unit 216 may record and analyze the history of alerts that have occurred. The alert history can include information such as the date and time the alert occurred, the names of the physicians and patients involved, and the reason for the possibility of misdiagnosis. Analyzing this information can be used to extract issues for improving diagnosis quality and provide feedback to physicians.

The alert methods exemplified above may be used alone or in combination. The selection of alert methods can be flexibly made according to the policy of the medical institution, the characteristics of the department, the proficiency of individual physicians, etc.

### Disclosure Items

This disclosure also includes the following configurations:

### [Item 1]

An information processing system comprising:
   an answer input unit configured to accept an answer from a patient to a question for differentiating a mental disorder;
   a video image acquisition unit configured to acquire a video image capturing the patient;
   a biometric reaction detection unit configured to analyze the video image to detect a change in biometric reaction of the patient; and
   an estimation unit configured to estimate the mental disorder that the patient suffers from by inputting the accepted answer and the detected change in biometric reaction into a learning model, wherein the learning model is trained on the answer, the change in biometric reaction, and the mental disorder.

### [Item 2]

The information processing system according to Item 1, comprising:
a diagnosis result input unit configured to accept a diagnosis result of the mental disorder of the patient by a medical professional based on the answer; and
a misdiagnosis possibility determination unit configured to determine whether the estimation result of the mental disorder by the estimation unit matches the diagnosis result or not.

### [Item 3]

The information processing system according to Item 1, wherein the estimation unit calculates probabilities that the patient suffers from each of a plurality of mental disorders.

### [Item 4]

The information processing system according to Item 1, wherein the video image acquisition unit acquires the video image capturing the patient engaging in daily conversation.

### [Item 5]

The information processing system according to Item 4, comprising:
a daily conversation processing unit configured to conduct daily conversation with the patient and generate second speech content to convey to the patient in response to first speech content from the patient;
wherein the video image acquisition unit acquires the video image capturing conversation between the patient and the daily conversation processing unit.

### [Item 6]

An information processing method comprising steps performed by a computer of:
accepting an answer from a patient to a question for differentiating a mental disorder;
acquiring a video image capturing the patient;
analyzing the video image to detect a change in biometric reaction of the patient; and
estimating the mental disorder that the patient suffers from by inputting the accepted answer and the detected change in biometric reaction into a learning model, wherein the learning model is trained on the answer, the change in biometric reaction, and the mental disorder.

### [Item 7]

A non-transitory computer-readable medium storing instructions that, when executed by one or more processors, cause the one or more processors to perform operations comprising:
accepting an answer from a patient to a question for differentiating a mental disorder;
acquiring a video image capturing the patient;
analyzing the video image to detect a change in biometric reaction of the patient; and
estimating the mental disorder that the patient suffers from by inputting the accepted answer and the detected change in biometric reaction into a learning model, wherein the learning model is trained on the answer, the change in biometric reaction, and the mental disorder.

### REFERENCE SIGNS LIST

1 physician terminal
2 management server
3 patient terminal

1. An information processing system comprising:
an answer input unit configured to accept an answer from a patient to a question for differentiating a mental disorder;
a video image acquisition unit configured to acquire a video image capturing the patient;
a biometric reaction detection unit configured to analyze the video image to detect a change in biometric reaction of the patient; and
an estimation unit configured to estimate the mental disorder that the patient suffers from by inputting the accepted answer and the detected change in biometric reaction into a learning model, wherein the learning model is trained on the answer, the change in biometric reaction, and the mental disorder.

2. The information processing system according to claim 1, comprising:
a diagnosis result input unit configured to accept a diagnosis result of the mental disorder of the patient by a medical professional based on the answer; and
a misdiagnosis possibility determination unit configured to determine whether the estimation result of the mental disorder by the estimation unit matches the diagnosis result or not.

3. The information processing system according to claim 1, wherein the estimation unit calculates probabilities that the patient suffers from each of a plurality of mental disorders.

4. The information processing system according to claim 1, wherein the video image acquisition unit acquires the video image capturing the patient engaging in daily conversation.

5. The information processing system according to claim 4, comprising:
a daily conversation processing unit configured to conduct daily conversation with the patient and generate second speech content to convey to the patient in response to first speech content from the patient; wherein
the video image acquisition unit acquires the video image capturing conversation between the patient and the daily conversation processing unit.

6. An information processing method comprising steps performed by a computer of:
accepting an answer from a patient to a question for differentiating a mental disorder;
acquiring a video image capturing the patient;
analyzing the video image to detect a change in biometric reaction of the patient; and
estimating the mental disorder that the patient suffers from by inputting the accepted answer and the detected change in biometric reaction into a learning model, wherein the learning model is trained on the answer, the change in biometric reaction, and the mental disorder.

7. A non-transitory computer-readable medium storing instructions that, when executed by one or more processors, cause the one or more processors to perform operations comprising:
accepting an answer from a patient to a question for differentiating a mental disorder;
acquiring a video image capturing the patient;
analyzing the video image to detect a change in biometric reaction of the patient; and
estimating the mental disorder that the patient suffers from by inputting the accepted answer and the detected change in biometric reaction into a learning model, wherein the learning model is trained on the answer, the change in biometric reaction, and the mental disorder.
